(19) United States
(12) Patent Application Publication
(10) Pub. No.: US 2006/0154970 A1
(43) Pub. Date: Jul. 13, 2006

(54) Alanines compounds, method of preparing them and their use

(76) Inventors: Yushe Yang, Shanghai (CN); Lei Tang, Shanghai (CN); Ruyun Ji, Shanghai (JP); Kaixian Chen, Shanghai (CN)

Correspondence Address: NIXON & VANDERHYE, PC, 901 NORTH GLEBE ROAD, 11TH FLOOR, ARLINGTON, VA, 22203, US

(21) Appl. No.: 10/543,091

(22) PCT Filed: Jan. 28, 2003
(86) PCT No.: PCT/CN03/00096

§ 371 Date: Jul. 22, 2005

(51) Int. Cl. A61K 31/44; A61K 31/428; A61K 31/423; A61K 31/421; A61K 31/198; A61K 31/405

(52) U.S. Cl. 514/352; 514/374; 514/375; 514/419; 514/563; 562/450; 548/495; 548/216; 548/236; 546/309

(57) ABSTRACT

The present invention provides alanines compounds of formula (I) or their salts:

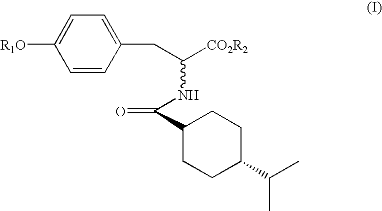

In the formula (I), the configuration of α-Carbon atom of alanine is R or S; R₁ is hydrogen, unsubstituted or substituted C₁₋₆alkyl, unsubstituted or substituted aryl or aromatic heterocyclic group; R₂ is hydrogen or unsubstituted or substituted C₁₋₆ alkyl. The present invention also provides two methods of preparing these compounds or their salts, and their use in preparing anti II type diabetic medicine.

### FIELDS OF INVENTION

The present invention relates to the fields of medicinal chemistry and endocrinotherapy, specifically to the synthesis of alanine compounds and their use in the preparation of drugs for type II diabetes.

### BACKGROUND OF THE ART

Type II diabetes is a metabolic disorder characterized by hyperglycemia (the fasting blood glucose concentration is above 130 mg/dL) and glucosuria. Long-term hyperglycemia often leads to various complications, such as neuropathy, retinopathy and nephropathy. Especially, the cardiovascular complications are the main causes of leading to disability and death in diabetic patients [Shinkai, H. Exp. Opin. Ther. Patents. 2000, 10: 596]. Therefore, controlling the blood glucose level in diabetic patients is very important for postponing or blocking the occurrence of the complications. Drugs currently available for clinically controlling the blood glucose level in the patients are mainly sulfonylureas for promoting the release of insulin and biguanides. Since insulin resistance is the predominant pathogenesis in type II diabetes, study on insulin-sensitizing agents has become an important direction in the development of anti-type II diabetes drugs. The first thiazolidinedione insulin-sensitizer troglitazone was marketed in 1997. This drug and another two thiazolidinediones, piglitazone and rosiglitazone which were released later, were reported to clinically control the blood glucose level in patients effectively. However, hepatic toxicity were found for these thiazolidinediones after marketing [Henry, R. R. Endocrinol. Metab. Clic. North Am. 1997, 26, 553], and troglitazone was even withdrawn from market for its higher hepatic toxicity. The toxicity of this type of compounds was suspected to be related to the thiazolidinedione group. The study of insulin-sensitizers has shifted to the synthesis and development of non-thiazolidinedione compounds for anti-type II diabetes treatment.

The references mentioned above and herein are incorporated herein in their entirety by reference.

One objective of the present invention is to provide novel alanine compounds having insulin-sensitizing activities and their pharmaceutical acceptable salts.

Another objective of the present invention is to provide preparative methods for the alanine compounds and their salts.

A further objective of the present invention is to provide the application of the alanine compounds and their salts in the preparation of drugs for type II diabetes.

Methods of treating type II diabetes with the compounds of the present invention are also described herein.

### SUMMARY OF THE INVENTION

The invention provides the alanine compounds represented by the following formula (I) and their salts:

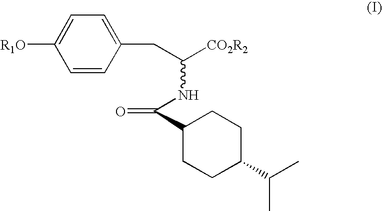

In the formula (I), the configuration of α-carbon atom of alanine is R or S. The present invention further provides R or S forms of the compounds of formula (I).

R₁ is hydrogen, unsubstituted or substituted C₁₋₆ alkyl, or unsubstituted or substituted aryl or aromatic heterocyclic group;

R₁ may be more specifically any one of the following:

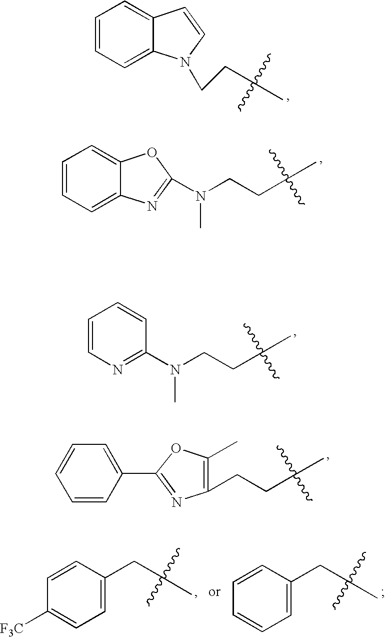

R₂ is hydrogen or unsubstituted or substituted C₁₋₆ alkyl.

The present invention also provides two preparative methods for the alanine compounds with the structure of formula (I) and their salts.

The first preparative method includes the following steps:

(1) Condensing trans-4-isopropylcyclohexylcarboxylic acid N-hydroxylsuccinimide ester (compound A) and L-tyrosine methyl ester or D-tyrosine methyl ester condense in an inert solvents to obtain 2-[N-(trans-4-isopropylcyclohexylcarbonyl)amino]-3-(4-hydroxyphenyl)propionic acid methyl ester (compound B);

(2) Conducting Mitsunobu reaction of Compound B and corresponding heterocycloalkyl alcohol or aromatic alcohol, followed by hydrolyzation with inorganic bases to obtain the compound of formula (I), wherein R₁ is; 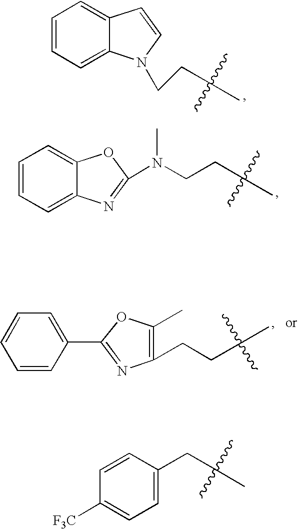
and R₂ is H; or

Conduct an etherification of compound B and corresponding alkyl halide under alkali condition to obtain the compound of formula (I), wherein R₁ is; 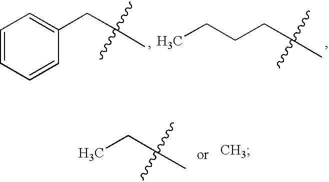
and R₂ is H; or

Hydrolyzing compound B to obtain the compound of formula (I), wherein R₁ and R₂ both are hydrogen;

(3) preparing the corresponding pharmaceutical acceptable salts in a manner known in the art.

The second preparative method includes the following steps:

(1) Condensing compound B with amino-protected 2-methylaminoethanol, then deprotects, and reacts with excessive 2-fluoropyridine under reflux, followed by hydrolyzation under basic conditions to give compounds of formula (I), wherein R₁ is 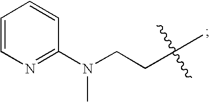
and R₂ is hydrogen; and

(2) preparing corresponding pharmaceutically acceptable salts according to methods known in the art.

The present invention further provides methods of preparing alanine compounds of formula (I) and their salts as drugs for type II diabetes as well as methods of treating type II diabetes by administration of the compounds of formula (I) to a mammal, such as a human or person in need of such treatment.

### DESCRIPTION OF THE INVENTION

Unless specified, the terms in the description have the following definitions The phrase “substituted or unsubstituted C₁₋₆ alkyl” includes the saturated or unsaturated, substituted or unsubstituted linear or branched alkane-derived radical containing 1 to 6 carbon atoms. Specific examples include methyl, ethyl, propyl, isopropyl, butyl, isobutyl, sec-butyl, tert-butyl, amyl, isoamyl, neo-pentyl, tert-amyl, 1-methylbutyl, 2-methylpropyl, hexyl, isohexyl, 1-methylamyl, 2-methylamyl, 3-methylamyl, 2-methylbutyl, 1,1-dimethylbutyl, 1,2-dimethylbutyl, 1,3-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl or the like. Among these groups, the alkyls of 1 to 4 carbon atoms, such as methyl, ethyl, propyl, isopropyl, butyl, or the like, are preferable. And methyl, ethyl, propyl are more preferable, methyl and ethyl are the most preferable.

The term “aryl” means an aromatic radical, such as an aryl of 6 to 14 carbon atoms, and including phenyl, tolyl, xylyl, biphenyl, naphthyl, indenyl, anthryl, phenanthryl, wherein phenyl and naphthyl are more preferable, and phenyl is the most preferable.

The term “aromatic heterocyclic group” means five or six-membered hetero aromatic radical containing 1, 2, 3 or hetero atoms selected from oxygen, nitrogen and sulfur, and including furyl, thienyl, pyrrolyl, imidazolyl, thiazolyl, pyrazolyi, isothiazolyl, isooxazolyl, pyridyl, pyrimidinyl, pyridazinyl, pyrazinyl, triazolyl, tetrazolyl, or the like. Among these groups, thienyl, furyl, oxazolyl, isooxazolyl and thiazolyl are preferable, and thienyl, oxazolyl and isooxazolyl are more preferable.

The term “substituted alkyl”, “substituted aryl” and “substituted aromatic heterocyclic group” mean that the above “alkyl”, “aryl” and “heteroaryl” can be optionally substituted by the groups selected from halogen atoms, alkyl, alkoxyl, acyloxy, —OH, —NH₂, —NO₂, —NHAc.

The “pharmaceutical acceptable salts” may specifically include the salts with inorganic acids, such as hydrochloric acid, hydrobromic acid, hydrofluoric acid, sulfuric acid, nitric acid, phosphoric acid or the like; the acid-addtion salts with organic acids, such as formic acid, acetic acid, propionic acid, oxalic acid, malonic acid, succinic acid, fumaric acid, maleic acid, lactic acid, malic acid, tartaric acid, citric acid, picric acid, methanesulfonic acid, ethylsulfonic acid or the like, or with acidic amino acids, such as aspartic acid, glutamic acid or the like; or the salts formed with alkalis, such as the salts with inorganic alkalis of Na, K, Ca, Al or the like, ammonium salt, methylamine salt, ethylamine salt, ethanolamine salt or the like; or the salts formed with basic amino acids, such as lysine, arginine, ornithine or the like.

The alanine compounds of formula (I) and their salts in the present invention are prepared as follows:

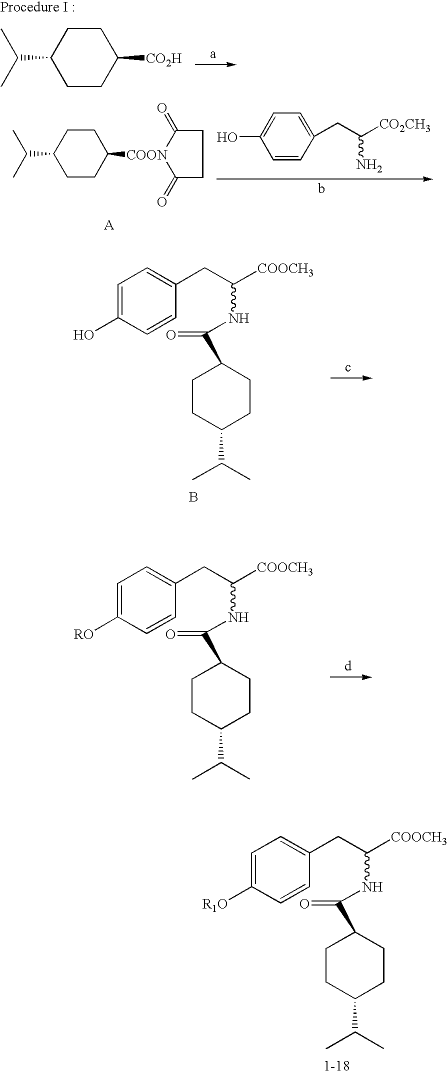

In the scheme: a. N-hydroxylsuccimide, Dicyclohexylcarbodiimide; b. chloroform; c. R₃—OH, triphenylphosphine, diethyl azodicarboxylate; or RX, K₂CO₃, DMF; d. 1N lithium hydroxide, tetrahydrofuran/methanol (3:1).

An embodiment of procedure I is as follows:

1. trans-4-isopropylcyclohexylcarboxylic acid (shinkai H. J. Med. Chem. 1989, 32, 1436-1441) reacts with N-hydroxysuccimide (HOSu) and N,N′-Dicyclohexylcarbodiimide (DCC) to dehydrate and obtain trans-4-isopropylcyclohexylcaboxylic acid N-hydroxysuccinimide ester (compound A). The compound A condense with L-tyrosine methyl ester or D-tyrosine methyl ester in an inert solvent such as chloroform, dichloromethane, ether, tetrahydrofuran under a temperature of −10° C.-50° C. for 0.1-72 h to obtain 2-[N-(trans-4-isopropylcyclohexylcarbonyl)amino]-3-(4-hydroxyphenyl)propionic acid methyl ester (compound B). The optimal reaction condition is to react in chloroform under room temperature for 24 h.

Compound B and corresponding heterocycloalkyl alcohol or aromatic alcohol conduct the Mitsunobu reaction, then is hydrolyzed with inorganic base to give compounds 1-8. The solvents used in Mitsunobu reaction are the anhydrous inert solvents, such as anhydrous tetrahydrofuran, anhydrous ether, chloroform, dichloromethane or the like. The reaction temperature is between 0-100° C., the reaction time is between 0.1-3 day. The inorganic bases suitable for hydrolyzation are sodium hydroxide, lithium hydroxide, potassium hydroxide, sodium carbonate, potassium carbonate, lithium carbonate, etc; the hydrolyzing temperatures is between −10-100° C.; the solvent is a mixture of tetrahydrofuran and methanol in different ratios, or a mixture of other inert solvents, such as chloroform, dichloromethane, benzene, and suitable alcohols, such as ethanol, propanol, isopropanol in different ratios. The optimal hydrolyzing condition is to hydrolyze with lithium hydroxide under room temperature for 24 h with tetrahydrofuran/methanol (3:1)) as solvent.

2. Compound B conduct etherification with corresponding alkyl halide under basic conditions to obtain compounds 9-16. The suitable inorganic bases for etherification reaction are sodium hydroxide, lithium hydroxide, potassium hydroxide, sodium carbonate, potassium carbonate, lithium carbonate, etc. The etherification temperature is between °10-180° C., The suitable solvents are dimethylformamide, DMSO, H₂O, etc. The reaction time is 1-72 h.

3. Compounds 17-18 are obtained directly by hydrolyzation of compound B.

4. Corresponding pharmaceutical acceptable salts are prepared according to requirement.

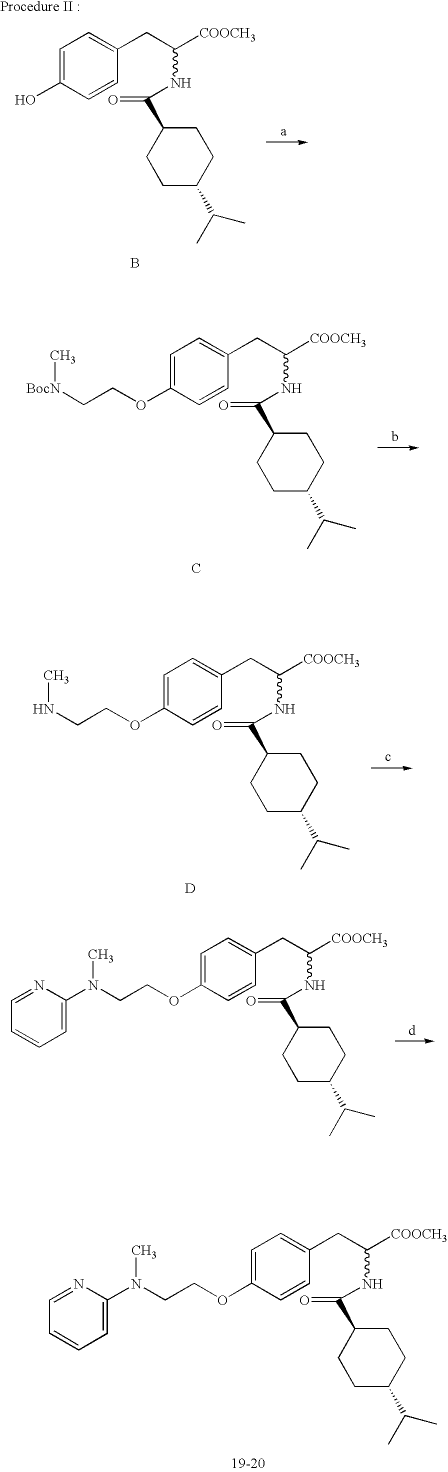

In the scheme: a. triphenylphosphine, diethyl azodicarboxylate, 2-(N-carbobenzoxy-N-methylamino)ethanol, tetrahydrofuran; b. trifluoracetic acid, dichloromethane; c. 2-fluoropyridine, reflux; d. 1N lithium hydroxide, tetrahydrofuran/methanol (3:1).

An embodiment of procedure II is as follows

1. Compound B is prepared by the same method as in Procedure I.

2. Compound B condenses with tert-butoxycarbonyl protecting 2-methylaminoethanol to obtain (2S)-2-[N-(trans-4-isopropylcyclohexylcarbonyl) amino]-3-[4-(N-methyl-N-tert-butoxycarbonylaminoethoxy)phenyl]propionic acid methyl ester (compound C). The solvent used in condensation reaction is the anhydrous inert solvent such as anhydrous tetrahydrofuran, anhydrous ether, chloroform, dichloromethane or the like. The reaction temperature is between 0-100° C. The reaction time is between 0.1-3 day. The compound C is treated with trifluoracetic acid under −10-50° C. for 1-72 h to remove the protective group and obtain (2S)-2-[N-(trans-4-isopropylcyclohexylcarbonyl)amino]-3-[4-(N-methylaminoethoxy)phenyl]propionic acid methyl ester (compound D). Then compound D is refluxed and condensed with excessive 2-fluoropyridine to obtain the ester precursors of compounds 19-20. Then compounds 19-20 are obtained by hydrolyzation with base. The suitable inorganic bases for hydrolyzation are sodium hydroxide, lithium hydroxide, potassium hydroxide, sodium carbonate, potassium carbonate, lithium carbonate, etc. The hydrolyzing temperatures is between −10-100° C. The hydrolyzing solvent is a mixture of tetrahydrofuran and methanol in different ratios, or a mixture of other inert solvents, such as chloroform, dichloromethane, benzene, and suitable alcohols, such as ethanol, propanol, isopropanol in different ratios. The optimal hydrolyzing condition is to hydrolyze with lithium hydroxide under room temperature for 24 h with tetrahydrofuran-methanol (3:1) as solvent.

3. Corresponding pharmaceutical acceptable salts are prepared according to requirement.

The representative alanine compounds of formula (I) according to the present invention are listed as follows:

(1) (2S)-2-[N-(trans-4-isopropylcyclohexylcarbonyl)amino]-3-[4-[2-(5-methyl-2-phenyl-4-oxazolyl)ethoxy]phenyl]propionic acid;

(2) (2S)-2-[N-(trans-4-isopropylcyclohexylcarbonyl)amino]-3-[4-[2-[N-methyl-N-(2-benzoxazolyl)amino]ethoxy]phenyl]propionic acid (3);

(3) (2R)-2-[N-(trans-4-isopropylcyclohexylcarbonyl)amino]-3-[4-[2-(5-methyl-2-phenyl-4-oxazolyl)ethoxy]phenyl]propionic acid (2);

(4) (2R)-2-[N-(trans-4-isopropylcyclohexylcarbonyl)amino]-3-[4-[2-[N-methyl-N-(2-benzoxazolyl)amino]ethoxy]phenyl]propionic acid;

(5) (2S)-2-[N-(trans-4-isopropylcyclohexylcarbonyl)amino]-3-[4-[2-(1-indolyl)ethoxy]phenyl]propionic acid;

(6) (2R)-2-[N-(trans-4-isopropylcyclohexylcarbonyl)amino]-3-[4-[2-(1-indolyl)ethoxy]phenyl]propionic acid;

(7) (2S)-2-[N-(trans-4-isopropylcyclohexylcarbonyl)amino]-3-[4-(4-trifluoromethylbenzyloxy)phenyl]propionic acid;

(8) (2R)-2-[N-(trans-4-isopropylcyclohexylcarbonyl)amino]-3-[4-(4-trifluoromethylbenzyloxy)phenyl]propionic acid;

(9) (2S)-2-[N-(trans-4-isopropylcyclohexylcarbonyl)amino]-3-(4-benzyloxyphenyl)propionic acid;

(10) (2R)-2-[N-(trans-4-isopropylcyclohexylcarbonyl)amino]-3-(4-benzyloxyphenyl)propionic acid;

(11) (2S)-2-[N-(trans-4-isopropylcyclohexylcarbonyl)amino]-3-(4-butoxyphenyl)propionic acid;

(12) (2R)-2-[N-(trans-4-isopropylcyclohexylcarbonyl)amino]-3-(4-butoxyphenyl)propionic acid;

(13) (2S)-2-[N-(trans-4-isopropylcyclohexylcarbonyl)amino]-3-(4-ethoxyphenyl)propionic acid;

(14) (2R)-2-[N-(trans-4-isopropylcyclohexylcarbonyl)amino]-3-(4-ethoxyphenyl)propionic acid;

(15) (2S)-2-[N-(trans-4-isopropylcyclohexylcarbonyl)amino]-3-(4-methoxyphenyl)propionic acid;

(16) (2R)-2-[N-(trans-4-isopropylcyclohexylcarbonyl)amino]-3-(4-ethoxyphenyl)propionic acid;

(17) (2S)-2-[N-(trans-4-isopropylcyclohexylcarbonyl)amino]-3-(4-hydroxyphenyl)propionic acid;

(18) (2R)-2-[N-(trans-4-isopropylcyclohexylcarbonyl)amino]-3-(4-hydroxyphenyl)propionic acid;

(19) (2S)-2-[N-(trans-4-isopropylcyclohexylcarbonyl)amino]-3-[4-[2-[N-methyl-N-(2-pyridyl)amino]ethoxyl]phenyl]propionic acid;

(20) (2R)-2-[N-(trans-4-isopropylcyclohexylcarbonyl)amino]-3-[4-[2-[N-methyl-N-(2-pyridyl)amino]ethoxyl]phenyl]propionic acid;

(21) (2S)-2-[N-(trans-4-isopropylcyclohexylcarbonyl)amino]-3-(4-hydroxyphenyl)propionic acid methyl ester; or

(22) (2R)-2-[N-(trans-4-isopropylcyclohexylcarbonyl)amino]-3-(4-hydroxyphenyl)propionic acid methyl ester.

The structure formulas of compounds 1-20 mentioned above see table 1. table 1

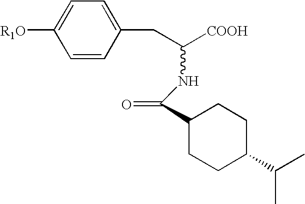

| No. | R₁ | Config.* |
|---|---|---|
| 1 2 | 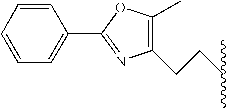 | S R |
| 3 4 | 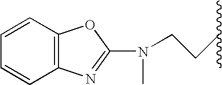 | S R |
| 5 6 | 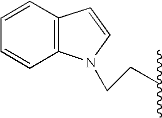 | S R |
| 7 8 | 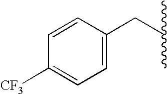 | S R |
| 9 10 | 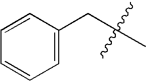 | S R |
| 11 12 | 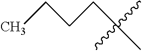 | S R |
| 13 | 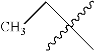 | S |
| 14 | 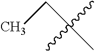 | R |
| 15 | CH₃ | S |
| 16 | CH₃ | R |
| 17 | H | S |
| 18 | H | R |
| 19 20 | 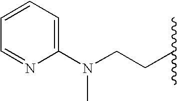 | S R |

*The configuration of α-carbon atom

Evaluation of Biological Activities:

Insulin-sensitizing agents can promote the differentiation of preadipocytes toward adipocytes, thus insulin-sensitizing agents could be identified according to the differentiation of pre-adipocytes. Following the methods reported in the literature [Kletzin B F. Mol. Pharm. 1991, 41, 393], the insulin-sensitizing activities of the compounds of formula (I) of the present invention were evaluated in 3T3-L1 pradipocyte model with triglyceride accumulation in cells as the indication of differentiation.

The 3T3-L1 preadipocytes were incubated in DMEM (Dulbecco's Modified Eagle's Medium) containing 10% NBS (newborn calf serum) and subcultured every 3 days. The calls were transferred to 24-pore plates, and after the pores were filled completely the cells were treated with IBMX (isobutylmethylxanthine) (0.5 mmol/L), DEX (dexamethasone) (1 μmol/L) and insulin (1.0 μmol/L) for 48 h. Different amounts of test compounds were incubated with the cells until the end of the experiment. Cells were collected and the contents of triglyceride and protein therein were determined by colorimetry. Enhancements of triglyceride in cells after drug-administrating were calculated.

The positive control group was rosiglitazone, and the solvent control group was a culturing medium containing 0.1% of DMSO. The enhancement of triglyceride in cells was determined in three different concentrations (0.01, 0.1, 1 μmol/L) of the tested compounds.

The insulin-sensitizing activity of the alanine compounds of the present invention are presented in table 2, from which it can be seen that Compound 1 and compound 2 have stronger insulin-sensitizing activity. Thus the alanine compounds of the present invention can be used to control the blood glucose level in type II diabetes patients and inhibit the occurrence of complications caused by the type II diabetes.

The alanine compounds of the present invention do not contain a thiazolidinedion group, but possess the similar insulin-sensitizing activity to that of the thiazolidinedion compounds. Therefore, it is possible that the compounds disclosed herein will be useful for treatment of type II diabetes and its complications.

TABLE 2
The Percentage of Triglyceride Increased in 3T3-L1 Cells

| | Concentrations^{a} (μ mol/L) | | |
|---|---|---|---|
| Compounds | 0.01 | 0.1 | 1 |
| 1 | 1.56 | 21.18 | 47.09 |
| 2 | 10.73 | 16.54 | 30.68 |
| 3 | −0.37 | 6.70 | 19.97 |
| 4 | −4.77 | 5.05 | 10.97 |
| 5 | −1.54 | −4.64 | −6.92 |
| 6 | ND | ND | ND |
| 7 | 11.75 | 5.80 | 4.68 |
| 8 | ND | ND | ND |
| 9 | 6.00 | 3.35 | −0.69 |
| 10 | ND | ND | ND |
| 11 | 0.00 | 0.36 | 2.23 |
| 12 | ND | ND | ND |
| 13 | 1.89 | 1.83 | −4.74 |
| 14 | ND | ND | ND |
| 15 | −0.21 | 5.99 | 5.04 |
| 16 | ND | ND | ND |
| 17 | 0.22 | 10.84 | 0.85 |
| 18 | ND | ND | ND |
| 19 | −1.46 | −2.24 | −1.62 |
| Rosiglitazone^{b} | 27.20 ± 2.35 | 34.93 ± 2.14 | 39.21 ± 2.27 |

^{a}Average data in 3 pores.
^{b}Sample number n = 22 ; mean ± SE.
ND: Not Done.

The compounds and their pharmaceutical acceptable salts of the present invention may be prepared into many forms of preparations, which contain a safe and effective dosage of the compounds or their pharmaceutical acceptable salts in the present invention, and the pharmaceutical acceptable carrier.

“safe and effective dosage” means the amount of the compounds that can improve the condition of patients but do not lead to serious side effects. The safe and effective dosage of compounds is determined according to the age, condition, and course of treatment of the subjects accepting the therapy and will usually be determinable by one of ordinary skill by routine experimentation.

“Pharmaceutical acceptable carrier” refers to one or many kinds of compatible solid or liquid stuffing materials or gel substances, which are suitable for human use and have enough purity and low toxicity. “compatible” is used to indicate that each component in the compositions can mixed with the compounds of the present invention and with each other without significantly reducing the effect of the compounds. Examples of the pharmaceutical acceptable carrier include cellulose and its derivatives (CMC-Na, EC—Na, cellulose acetatic acid ester etc.), gelatin, steatite, solid lubricants (such as stearic acid, magnesium stearate), CaSO₄, vegetable oils (such ad soya oil, sesame oil, peanut oil, olive oil etc.), polybasic alcohol (such as propylene glycerin, mannitol, sorbierite etc.), emulsifying agent (such as tweens®), moistening agent (such as sodium dodecylsulfate), coloring agent, flavoring agent, stabilizer, antioxidant, antiseptic, pyrogen-free water etc.

### THE PREFERABLE EMBODIMENTS OF THE INVENTION

The present invention will be further explained with reference to the following examples, but they don't limit the present invention in any way. In all examples, the melting points were measured with MEL-TEMP melting point apparatus and the thermomoter was not calibrated; ¹H NMR spectra were recorded on a Varian Mercury 400 NMR spectrometer, the chemical shifts are expressed as δ (ppm); silica gel for separation id 200-300 mesh unless otherwise specified.

### EXAMPLE 1

### (2S)-2-[N-(trans-4-isopropylcyclohexylcarbonyl)amino]-3-[4-[2-(5-methyl-2-phenyl-4-oxazole)ethoxy]phenyl]propionic acid(1);

(1) condensation: 0.605 g(2.31 mmol) of triphenylphosphine was added into a solution of 0.313 g (1.54 mmol) of 2-(5-methyl-2-phenyl4-oxazole)ethanol and 0.535 g (1.54 mmol) of (2S)-2-[N-(trans-4-isopropylcyclohexylcarbonyl)amino]-3-(4-hydroxyphenyl)propinia acid methyl ester ((S)—B) in 30 ml of anhydrous tetrahydrofuran, then 370 μl (2.31 mmol) of diethyl azodicarboxylate was added dropwise at 0° C. The resulting solution was stirred at room temperature for 24 h. After solvent was removed under reduced pressure, the residue was diluted with ether and a solid separated out, the white solid was filtered out and recrystallized with methanol to give 0.44 g of (2S)-2-[N-(trans-4-isopropylcyclohexylcarbonyl)amino]-3-[4-[2-(5-methyl-2-phenyl-4-oxazole)ethoxy]phenyl]propionic acid methyl ester, yield: 53.8%.

(2) hydrolyzation: 0.26 g (0.5 mmol) of the resultant in step (1) was dissolved in 1 ml of mixed solvent of tetrahydrofuran-methanol (3:1), then 1 ml of 1N solution of LiOH in H₂O was added, the resulting solution was stirred at room temperature for 24 h. The pH of the solution was adjusted to 5 with 1N HCl, and 5mL of H₂O was added, then this mixture was stirred and filtered, the filter cake was recrystallizated with methanol to give 0.2 g of the captioned compound, yield: 77.2%. m.p. 151-153° C. (dec). [α]_{D}²⁵ 81.1(c, 0.545, CHCl₃).

¹H NMR (DMSO-d₆): δ=0.80(d, J=7.6 Hz, 6H, isoproply-CH₃), 0.85-1.0(m, 3H, isoproply-CH, cyclohexyl-CH₂—), 1.10-1.40(m, 3H, cyclohexyl-CH₂—, —CH—), 1.50-1.72(m, 4H, cyclohexyl-CH₂—), 2.00(m, 1H, cyclohexyl-CH—), 2.35(s, 3H, oxazolyl-CH₃), 2.75(dd, J=14 Hz, 9.9 Hz, 1H, Ph-CH—), 2.90(t, J=6.6 Hz, 2H, oxazolyl-CH₂—), 2.96(dd, J=14 Hz, 4.7 Hz, 1H, Ph-CH—), 4.17(t, J=6.6 Hz, 2H, —CH₂O—), 4.21(m, 1H, —CHCOO—), 6.81(d, J=8.8 Hz, 2H, Ph-H), 7.13(d, J=8.8 Hz, 2H, Ph-H), 7.50(m, 3H, Ph-H), 7.92(m, 3H, Ph-H, —NH), 12.6(s, 1H, —COOH); ¹³C NMR (CDCl₃): δ=10.0, 19.9, 25.8, 28.8, 29.2, 29.5, 32.6, 36.1, 43.0, 45.9, 53.0, 66.2, 114.3, 126.2, 127.0, 128.0, 128.5, 130.3, 130.4, 132.1, 145.9, 157.8, 160.0, 175.8, 176.3.

Elements Analysis, C₃₁H₃₈N₂O₅ (518):
Calculated C, 71.81; H, 7.34; N, 5.41.
Found C, 71.49; H, 7.24; N, 5.36.
IR (KBr): 3282.3, 2933.2, 1710.6, 1631.5, 1554.4, 1513.9, 1251.6, 684.6 cm⁻¹; EI-MS(m/z): 518(1, M⁺), 186(100); HRMS: 518.2772 (C₃₁H₃₈N₂O₅).

### EXAMPLE 2

### (2R)-2-[N-(trans-4-isopropylcyclohexylcarbonyl)amino]-3-[4-[2-(5-methyl-2-phenyl-4-oxazolyl)ethoxy]phenyl]propionic acid(2)

With (2R)-2-[N-(trans-4-isopropylcyclohexylcarbonyl)amino]-3-(4-hydroxy-phenyl)propionic acid methyl ester ((R)—B) as starting material, the captioned compound was prepared by the same method as in example 1. m.p. 151-153° C.(dec). [α]_{D}²⁵ −82.5 (c, 0.217, CHCl₃). ¹H NMR is the same as that of the captioned compound in example 1.

Elements Analysis, C₃₁H₃₈N₂O₅ (518):
Calculated C, 71.81; H, 7.34; N, 5.41.
Found C, 71.40; H, 8.16; N, 5.33.

IR(KBr): 3282.3, 2933.2, 2854.2, 1712.5, 1633.4, 1554.4, 1513.9, 1249.7, 1176.4, 715.5, 686.5 cm⁻¹.

### EXAMPLE 3

### (2S)-2-[N-(trans-4-isopropylcyclohexylcarbonyl)amino]-3-[4-[2-[N-methyl-N-(2-benzoxazolyl)amino]ethoxy]phenyl]propionic acid (3)

A solution of 11 g (0.21 mmol) of compound D in 2 ml of tetrahydrofuran was added with 240 μl (0.51 mmol) of triethylamine and 40 mg (0.26 mmol) of 2-chlorobenzoxazole. The resulting solution was stirred at room temperature for 24 h. Tetrahydrofuran was removed under reduced pressure, the residue was mixed with 4 ml of ethyl acetate, then added 4 ml of saturated solution of NaHCO₃ in H₂O and stirred. Remained standing and separated the ethyl acetate layer, dried with anhydrous Na₂SO₄, and the residue was mixed with a mixed solvent of petrolether/ethyl acetate (1:1) to separate out a white solid. The solid was hydrolyzed with LiOH to give 0.042 g of the target compound. Yield: 39.6%. m.p. 179-180° C.(dec). [α]_{D}²⁵ 81.1 (c, 0.535, CHCl₃).

¹H NMR(DMSO): δ=0.81(d, J=6.9 Hz, 6H), 0.80-1.0(m, 3H), 1.15-1.40(m, 3H), 1.60(m, 4H), 1.95(m, 1H), 2.78(dd, J=13.5 Hz, 7.3 Hz, 1H), 2.96(dd, J=13.5 Hz, 4.8 Hz, 1H), 3.20(s, 3H), 3.82(t, J=5.5 Hz, 2H), 4.10(m, 1H), 4.20(t, J=5.5 Hz, 2H), 6.78(d, J=8.4 Hz, 2H), 6.95(t, J=7.7 Hz, 1H), 7.00(d, J=8.4 Hz, 2H), 7.10(t, J=7.0 Hz, 1H), 7.28(d, J=8.3 Hz, 1H), 7.35(d, J=8.0 Hz, 1H), 7.40(d, J=7.3 Hz, 1H).

Elements Analysis, C₂₉H₃₇N₃O₅ (507):
Calculated C, 68.64; H, 7.30; N, 8.28.
Found C, 68.45; H, 7.49; N, 8.21;

IR(KBr): 3322.8, 2935.2, 2865.7, 1731.8, 1648.9, 1589.1, 1513.9, 1465.7, 1247.7, 1250.3, 925.7, 740.5 cm⁻¹; EI-MS(m/z): 507(12, M⁺), 148(100).

### EXAMPLE 4

### (2R)-2-[N-(trans-4-isopropylcyclohexylcarbonyl)amino]-3-[4-[2-[N-methyl-N-(2-benzoxazolyl)amino]ethoxy]phenyl]propionic acid (4);

Using (R)—B as starting material, the captioned compound was prepared by the same method in example 3. m.p. 179-180° C. ¹H NMR is the same as that of the captioned compound in example 3.

Elements Analysis, C₂₉H₃₇N₃O₅ (507):
Calculated C, 68.64; H, 7.30; N, 8.28.
Found C, 68.64; H, 7.22; N, 8.08;

EI-MS(m/z): 507(6, M⁺), 148(100).

### EXAMPLE 5

### (2S)-2-[N-(trans-4-isopropylcyclohexylcarbonyl)amino]-3-[4-[2-(1-indolyl)ethoxy]phenyl]propionic acid (5)

Using 2-(1-indolyl)ethanol and compound (S)—B as starting materials, following the procedure as in example 1, the residue after removal of tetrahydrofuran was mixed with methanol and then separated out a solid. The target compound was obtained by hydrolyzation of the solid with LiOH. Yield: 39.6%.

¹H NMR(CDCl₃): δ=0.81(d, J=6.9 Hz, 6H), 0.80-1.10(m, 3H), 1.30-1.45(m, 3H), 1.70-1.90(m, 4H), 1.99(m, 1H), 3.07(dd, J=14.3 Hz, 6.2 Hz, 1H), 3.15(dd, J=14.3 Hz, 5.2 Hz, 1H), 4.23(t, J=5.5 Hz, 2H), 4.50(t, J=5.5 Hz, 2H), 4.75(m, 1H), 5.89(d, J=6.6 Hz, 1H), 6.50(d, J=3.9 Hz, 1H), 6.76(d, J=8.4 Hz, 2H), 7.00(d, J=8.4 Hz, 2H), 7.11(t, J=7.3 Hz, 1H), 7.24(m, 2H), 7.40(d, J=8.1 Hz, 1H), 7.62(d, J=7.7 Hz, 1H);

IR(KBr): 3305.4, 2933.2, 2863.8, 1712.5, 1650.8, 1513.9, 1463.7, 1242.0, 742.5 cm⁻¹; EI-MS(m/z): 476(11, M⁺), 144(100); HRMS: 476.2670 (C₂₉H₃₆N₂O₄).

### EXAMPLE 6

### (2R)-2-[N-(trans-4-isopropylcyclohexylcarbonyl)amino]-3-[4-[2-(1-indolyl)ethoxy]phenyl]propionic acid (6)

using (R)—B as starting materials, the captioned compound was prepared following the same procedure as in example 5. ¹H NMR is the same as that of the captioned compound in example 5.

IR(KBr): 3291.9, 2933.2, 2863.8, 1712.5, 1650.8, 1513.9, 1463.7, 1242.0, 742.5 cm⁻¹.

### EXAMPLE 7

### (2S)-2-[N-(trans-4-isopropylcyclohexylcarbonyl)amino]-3-[4-(4-trifluoromethylbenzyloxy)phenyl]propionic acid (7)

Using 4-trifluoromethylbenzyl alcohol and compound (S)—B as starting materials, ethyl ether as solvent, the captioned compound was prepared following the procedure as of compound 21. Yield: 60.6%. m.p. 171-172° C. [α]_{D}²⁵ 53.9 (c, 0.285, CHCl₃).

¹H NMR(CDCl₃): δ=0.81(d, J=6.7 Hz, 6H), 1.0(m, 3H), 1.40(m, 3H), 1.70-1.90(m, 4H), 2.05(m, 1H), 3.08(dd, J=5.5 Hz, 14.1 Hz, 1H), 3.19(dd, J=5.5 Hz, 14.3 Hz, 1H), 4.80(m, 1H), 5.08(s, 2H), 5.98(d, J=6.4 Hz, 1H), 6.88(d, J=8.2 Hz, 2H), 7.08(d, J=8.2 Hz, 2H), 7.52(d, J=8.0 Hz, 2H), 7.62(d, J=8.2 Hz, 2H).

Elements Analysis, C₂₇H₃₂F₃NO₄.1/2H₂O (500):
Calculated C, 64.80; H, 6.60; N, 2.80.
Found C, 65.20; H, 6.43; N, 3.01.

IR(KBr): 3328.6, 2931.3, 1731.8, 1612.2, 1511.9, 1328.7, 1243.9, 1166.7, 1124.3, 1068.4, 1018.2, 827.3 cm⁻¹; EI-MS(m/z): 491(3, M⁺), 159(100).

### EXAMPLE 8

### (2R)-2-[N-(trans-4-isopropylcyclohexylcarbonyl)amino]-3-[4-(4-trifluoromethylbenzyloxy)phenyl]propionic acid (8)

Using (R)—B as starting materials, the captioned compound was prepared following the same procedure as in example 7. m.p. 171-172° C. ¹H NMR is the same as that of the compound 7.

EI-MS(m/z): 491(10, M⁺), 159(100); HRMS: 491.2260(C₂₇H₃₂F₃NO₄).

### EXAMPLE 9

### (2S)-2-[N-(trans-4-isopropylcyclohexylcarbonyl)amino]-3-(4-benzyloxyphenyl)propionic acid (9)

A solution of 0.137 ml (1.14 mmol) of benzyl bromide and 0.13 g (0.38 mmol) of (2S)-2-[N-(trans-4-isopropylcyclohexylcarbonyl)amino]-3-(4-hydroxyphenyl)propionic acid methyl ester (compound (S)—B) in 1 ml N,N-dimethylformamide (DMF) was added with 0.16 g (1.14 mmol) of levigated K₂CO₃. The resulting mixture was stirred at 70° C. for 12 h. After cooling to 0° C., 5 ml of H₂O was added, the resulting solution was extracted with ethyl acetate (5 ml×2). The combined extract was washed with H₂O, dried with anhydrous Na₂SO₄. Solvent was removed under reduced pressure, the residue mixed with ether, and a white solid separated. The solid was hydrolyzed with LiOH to give 0.10 g of the target compound. Yield: 62.5%. m.p. 140-142° C.(dec). [α]_{D}70.9 (c, 0.83, CHCl₃).

¹H NMR(CDCl₃): δ=0.81(d, J=6.9 Hz, 6H), 1.0(m, 3H), 1.40(m, 3H), 1.70-1.90(m, 4H), 2.05(m, 1H), 3.04(dd, J=5.5 Hz, 13.9 Hz, 1H), 3.19(dd, J=5.5 Hz, 13.9 Hz, 1H), 4.81(m, 1H), 5.01(s, 2H), 6.12(d, J=7.3 Hz, 1H), 6.91(d, J=8.4 Hz, 2H), 7.08(d, J=8.4 Hz, 2H), 7.39(m, 5H).

Elements Analysis, C₂₆H₃₃NO₄.1/2H₂O (432):
Calculated C, 72.22; H, 7.87; N, 3.24.
Found C, 72.34; H, 7.71; N, 3.47;

IR(KBr): 3328.6, 2929.4, 2852.2, 1731.8, 1648.9, 1529.3, 1513.9, 1452.2, 1243.9, 1178.3, 1027.9, 732.8, 694.3 cm⁻¹; EI-MS(m/z): 423(4, M⁺), 254(24), 91(100).

### EXAMPLE 10

### (2R)-2-[N-(trans-4-isopropylcyclohexylcarbonyl)amino]-3-(4-benzyloxyphenyl)propionic acid (10)

using (R)—B as starting materials, the captioned compound was prepared following the same procedure as in example 9. m.p. 140-142° C.(dec). ¹H NMR is the same as that of the compound 9.

Elements Analysis, C₂₆H₃₃NO₄.1/2H₂O (432):
Calculated C, 72.22; H, 7.87; N, 3.24.
Found C, 72.36; H, 7.91; N, 3.32.

IR(KBr): 3326.7, 2929.4, 2852.2, 1729.9, 1648.9, 1529.3, 1513.9, 1452.2, 1243.9, 1178.3, 1027.9, 732.8, 694.3 cm⁻¹; EI-MS(m/z): 423(12, M⁺), 254(100).

### EXAMPLE 11

### (2S)-2-[N-(trans-4-isopropylcyclohexylcarbonyl)amino]-3-(4-butoxyphenyl)propionic acid (11)

Using n-bromobutane and compound (S)—B as starting materials, the captioned compound was prepared following the similar synthesis procedure as in example 9.

Yield: 70.3%. m.p. 120-121° C. [α]_{D} 87.9 (c, 1.145, CHCl₃).

⁻H NMR(CDCl₃): δ=0.82(d, J=6.9 Hz, 6H), 0.96(m, 6H), 1.38(m, 3H), 1.50(m, 2H), 1.75-1.90(m, 6H), 2.05(m, 1H), 3.04(dd, J=5.8 Hz, 14.3 Hz, 1H), 3.19(dd, J=5.5 Hz, 14.2 Hz, 1H), 3.91(t, J=6.6 Hz, 2H), 4.80(m, 1H), 5.99(d, J=7.3 Hz, 1H), 6.81(d, J=8.4 Hz, 2H), 7.07(d, J=8.4 Hz, 2H).

Elements Analysis, C₂₃H₃₅NO₄.1/3H₂O (395):
Calculated C, 69.87, H, 9.03, N, 3.54.
Found C, 69.60, H, 8.88, N, 3.64.

IR(KBr): 3305.4, 2933.2, 2869.6, 1716.4, 1646.9, 1544.7, 1513.9, 1245.8, 1178.3, 827.3 cm⁻¹; EI-MS(m/z): 389(7, M⁺), 163(100).

### EXAMPLE 12

### (2R)-2-[N-(trans-4-isopropylcyclohexylcarbonyl)amino]-3-(4-butoxyphenyl)propionic acid (12)

Using n-bromobutane and compound (R)—B as starting materials, the captioned compound was prepared following the similar synthesis procedure as in example 9. m.p. 120-121° C. ¹H NMR is the same as that of the compound 11.

Elements Analysis, C₂₃H₃₅NO₄ (389):
Calculated C, 70.95; H, 9.00; N,3.60.
Found C, 71.05; H, 9.11; N, 3.93.

IR(KBr) :3320.9, 2869.6, 2933.2, 1731.8, 1652.7, 1612.2, 1544.7, 1511.9, 1243.9, 827.3 cm⁻¹; EI-MS(m/z): 389(7, M⁺), 163(67), 220(100).

### EXAMPLE 13

### (2S)-2-[N-(trans-4-isopropylcyclohexylcarbonyl)amino]-3-(4-ethoxyphenyl)propionic acid (13)

Using ethyl bromide and compound (S)—B as starting materials, the captioned compound was prepared following the procedure as in example 9. Yield: 75.0%. m.p. 168-170° C. [α]_{D}²⁵ 93.2 (c, 1.13, CHCl₃).

¹H NMR(CDCl₃): δ=0.81(d, J=6.9 Hz, 4H), 1.0(m, 3H), 1.40(m, 5H), 1.75-1.90(m, 4H), 2.05(m, 1H), 3.04(dd, J=5.5 Hz, 14.0 Hz, 1H), 3.17(dd, J=5.1 Hz, 14.3 Hz, 1H), 4.00(m, 2H), 4.80(m, 1H), 5.97(d, J=7.6 Hz, 1H), 6.81(d, J=8.4 Hz, 2H), 7.03(d, J=8.3 Hz, 2H).

Elements Analysis, C₂₁H₃₁NO₄.1/3H₂O (367):
Calculated C, 68.66; H, 8.63; N, 3.81.
Found C, 68.93; H, 8.43; N, 3.86.

IR(KBr): 3303.5, 2935.2, 2958, 1724.1, 1708.6, 1643.1, 1542.8, 1513.9, 1442.5, 1247.7, 1215, 1176.4, 1051, 525.4 cm⁻¹; EI-MS(m/z): 361(16, M⁺), 192(100).

### EXAMPLE 14

### (2R)-2-[N-(trans-4-isopropylcyclohexylcarbonyl)amino]-3-(4-ethoxyphenyl)propionic acid (14)

Using ethyl bromide and compound (R)—B as starting materials, the captioned compound was prepared following the systhesis procedure in example 9. m.p. 168-170° C. ¹H NMR is the same as that of the compound 13.

Elements Analysis, C₂₁H₃₁NO₄ (361):
Calculated C, 69.81; H, 8.59; N, 3.87.
Found C, 69.40; H, 8.32; N, 3.90.

IR(KBr): 3305.4, 2933.2, 1706.7, 1641.2, 1540.9, 1513.9, 1444.4, 1249.7, 1215, 1049.1, 925.7, 804.2 cm⁻¹.

### EXAMPLE 15

### (2S)-2-[N-(trans-4-isopropylcyclohexylcarbonyl)amino]-3-(4-methoxyphenyl)propionic acid (15)

Using methyl iodide and compound (S)—B as starting materials, the captioned compound was prepared following the systhesis procedure in example 9. Yield: 67.1%. m.p. 149-151° C. [α]_{D}²⁵ 96.5 (c, 0.665, CHCl₃).

¹H NMR(CDCl₃): δ=0.85(d, J=6.6 Hz, 6H), 1.00(m, 3H), 1.40(m, 3H), 1.71-1.90(m, 4H), 2.05(m, 1H), 3.10(dd, J=5.9 Hz, 14.0 Hz, 1H), 3.17(dd, J=5.5 Hz, 13.9 Hz, 1H), 3.80(s, 3H), 4.80(m, 1H), 5.98(d, J=7.6 Hz, 1H), 6.81(d, J=8.8 Hz, 2H), 7.03(d, J=8.8 Hz, 2H).

Elements Analysis, C₂₀H₂₉NO₄ (347):
Calculated C, 69.16; H, 8.36; N, 4.03.
Found C, 69.06; H, 8.54; N, 4.14.

IR (KBr): 3280.4, 2937.1, 2860, 1720.2, 1646.9, 1542.8, 1515.8, 1440.6, 1249.7, 1215, 1031.7, 829.3 cm⁻¹; EI-MS(m/z): 347(1, M⁺), 121(100).

### EXAMPLE 16

### (2R)-2[N-(trans-4-isopropylcyclohexylcarbonyl)amino]-3-(4-ethoxyphenyl)propionic acid (16)

Using methyl iodide and compound (R)—B as starting materials, the captioned compound was prepared following the similar systhesis procedure in example 9. m.p. 149-151° C. ¹H NMR is the same as that of compound (S)-15.

Elements Analysis, C₂₀H₂₉NO₄.1/3H₂O(353):
Calculated C, 67.99; H, 8.41; N, 3.97.
Found C, 68.44; H, 8.08; N, 4.07.

IR (KBr): 3274.6, 2939, 2860, 1720, 1645, 1552.4, 1513.9, 1440, 1249.7, 1216.9, 1031.7, 829.3 cm⁻¹; EI-MS(m/z): 347(16, M⁺), 121(100).

### EXAMPLE 17

### (2S)-2-[N-(trans-4-isopropylcyclohexylcarbonyl)amino]-3-(4-hydroxyphenyl)propionic acid (17)

The target compound was obtained by hydrolyzation of (S)—B with LiOH. Yield: 81.1%. m.p. 156-157° C. [α]_{D}²⁵ 76.4 (c, 0.845, CHCl₃). ¹H NMR(DMCO-d₆): δ=0.81(d, J=7.0 Hz, 6H), 1.0(m, 3H), 1.40(m, 3H), 1.75-1.90(m, 4H), 2.15(m, 1H), 2.90(dd, J=8.1 Hz, 13.9 Hz, 1H), 3.10(dd, J=5.1 Hz, 13.9 Hz, 1H), 4.82(m, 1H), 6.72(d, J=8.4 Hz, 2H), 7.05(d, J=8.4 Hz, 2H).

Elements Analysis, C₁₉H₂₇NO₄.2H₂O(369):
Calculated C, 61.79; H, 8.40; N, 3.79.
Found C, 61.94; H, 8.17; N, 3.84.

IR (KBr): 3303.5, 2937.1, 2861.9, 1619.9, 1546.7, 1517.7, 1446.4, 1232.3, 827.3 cm⁻¹; EI-MS(m/z): 333(10, M⁺), 170(100), 107(84).

### EXAMPLE 18

### (2R)-2-[N-(trans-4-isopropylcyclohexylcarbonyl)amino]-3-(4-hydroxyphenyl)propionic acid (18)

The captioned compound was obtained by hydrolyzation of (R)—B with LiOH. m.p. 156-157° C. ¹H NMR is the same as that of compound 17.

Elements Analysis, C₁₉H₂₇NO₄.2/3H₂O(345):
Calculated C, 66.09; H, 8.21; N, 4.06.
Found C, 65.57; H, 8.71; N, 4.03.

IR(KBr): 3309.3, 2939.0, 2861.9, 1745.3, 1726.0, 1619.9, 1517.7, 1548.6, 1444.4, 1230.4, 825.4, 680.8, 538.1 cm⁻¹; EI-MS(m/z): 333(10, M⁺), 170(100), 107(54).

### EXAMPLE 19

### (2S)-2-[N-(trans-4-isopropylcyclohexylcarbonyl)amino]-3-[4-[2-[N-methyl-N-(2-pyridyl)amino]ethoxyl]phenyl]propionic acid (19)

A solution of 0.19 g (1.1 mmol) of 2-[(N-tert-butoxycarbonyl)-methylamino]ethanol and 0.347 g (1 mmol) of (S)—B in 15 ml of anhydrous tetrahydrofuran was added with 0.38 g (1.5 mmol) of triphenylphosphine, then 240 μl (1.5 mmol) of diethyl azodicarboxylate was added dropwise at 0° C. The resulting solution was stirred at room temperature for 24 h. The solvent was removed under reduced pressure, and the residue was chromatographed over silica gel H column with petroleum/ethyl acetate (2:1) as eluent to obtain 0.21 g of a colorless syrupy, yield: 41.6%.

0.21 g (0.42 mmol) of the colorless syrupy obtained in last step was dissolved in 4.8 ml of dichloromethane, then 4.8 ml of trifluoroacetic acid was added. The resulting solution was stirred at room temperature for 1 h. Then a part of the solvent were removed under reduced pressure at room temperature. The residual solution was neutralized with saturated NaHCO₃ and extracted with dichloromethane (10 ml×2). The combined organics were washed with H₂O, dried on Mg₂SO₄, filtered and concentrated in vacuo. The solvent was evaporated off from the filtrate. The residue was refluxed with 2 ml of 2-fluoropyridine for 24 h. Excessive 2-fluoropyridine was removed under reduced pressure, the residue was dissolved in small amount of acetone, then chromatographed over silica gel H column with acetone/petroleum (1:2) as eluent to obtain 0.081 g of white solid. Hydrolyzation of the white solid following the same procedure in example 1 provided 0.06 g of the captioned compound. Yield: 76.8%. m.p. 158-160° C. [α]_{D}²⁵ 30.0 (c, 0.26, CHCl₃).

¹H NMR (CDCl₃): δ=0.80 (d, J=7.6 Hz, 6H), 0.80-1.00(m, 3H), 1.13-1.40(m, 3H), 1.62-1.82(m, 4H), 1.96(m, 1H), 3.03(d, J=4.4 Hz, 2H), 3.10(s, 3H), 3.88(t, J=6.5 Hz), 3.98(t, J=6.6 Hz), 4.62(m, 1H), 5.40(s, 1H), 6.21(d, J=6.9 Hz, 1H), 6.55(m, 1H), 6.75(d, J=8.5 Hz, 2H), 6.98(d, J=8.5 Hz, 2H), 7.48(m, 1H), 8.03(m, 1H).

Elements Analysis, C₂₇H₃₇N₃O₄.1/2H₂O(476):
Calculated C, 68.07; H, 7.98; N, 8.82.
Found C, 67.86; H, 7.75; N, 8.79.

IR (KBr): 3299.7, 2931.3, 1720.2, 1637.3, 1608.4, 1511.9, 1425.2, 1247.7, 1213, 1000.9, 763.7 cm⁻¹; EI-MS(m/z): 467(2, M⁺), 121(98), 135(100).

### EXAMPLE 20

### (2R)-2-[N-(trans-4-isopropylcyclohexylcarbonyl)amino]-3-[4-[2-[N-methyl-N-(2-pyridyl)amino]ethoxyl]phenyl]propionic acid (20)

The captioned compound was obtained by the same procedure as in example 19 using (R)—B as starting material. m.p. 154-155° C. [α]_{D}²⁵ −30.5(c, 0.19, CHCl₃). ¹H NMR is the same as that of compound 19. IR (KBr): 3315.1, 2921.7, 2854.2, 1594.9, 1502.3, 1421.3, 1247.7, 765.6 cm⁻¹.

### EXAMPLE 21

### (2S)-2-[N-(trans-4-isopropylcyclohexylcarbonyl)amino]-3-(4-hydroxyphenyl)propionic acid methyl ester ((S)—B)

A solution of 0.7 g (4.1 mmol) of trans-4-isopropylcyclohexylcarboxylic acid and 0.53 g (4.6 mmol) of HOSu in 14 ml of chloroform was added 0.95 g (4.6 mmol)of DCC batchwise. The resulting solution was stirred at room temperature for 3 h. The solid produced was filtered off. 0.4 ml of glacial acetic acid was added into the filtrate and stirred at room temperature. After 2 h, 10 ml of saturated aqueous solution of NaHCO₃ was added into, stirred, and the water layer was removed. The chloroform layer was washed with 5 ml of H₂) and saturated saline solution, then dried over anhydrous Mg₂SO₄ and filtered under reduced pressure. 0.80 g (4.1 mmol) of L-tyrosine methyl ester was added, then stirred at room temperature for 24 h. The solution was washed with 1N HCl and water, dried with anhydrous Mg₂SO₄ and filtered under reduced pressure. After removal of the solvent under reduced pressure, the residue was dissolved in methanol, stand at −20° C., a white solid was separated out. The mother liquor was concentrated and then another portion of solid was collected. 0.53 g of white captioned compound was obtained together. Yield: 37.8%.

¹H NMR(CDCl₃): δ=0.82(d, J=6.9 Hz, 6H), 0.89-1.12(m, 3H), 1.30-1.43(m, 3H), 1.70-1.90(m, 4H), 2.05(m, 1H), 2.96(dd, J=6.5 Hz, 14.2 Hz, 1H), 3.09(dd, J=5.5 Hz, 14.7 Hz, 1H), 3.72(s, 3H), 4.85(m, 1H), 6.00(d, J=8.1 Hz, 1H), 6.70(d, J=8.4 Hz, 2H), 6.90(d, J=8.2 Hz, 2H).

### EXAMPLE 22

### (2R)-2-[N-(trans-4-isopropylcyclohexylcarbonyl)amino]-3-(4-hydroxyphenyl)propionic acid methyl ester ((R)—B)

(R)—B was obtained with D-tyrosine methyl ester as starting material by following the above operations.

1-9. (canceled)

10. An alanine compound of formula (I) or their salts: 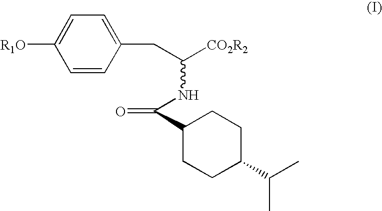
Wherein the configuration of a-carbon atom of alanine is R or S;
R₁ is hydrogen, substituted or unsubstituted C₁₋₆ alkyl, or aryl or aromatic heterocyclic group selected from the following groups: 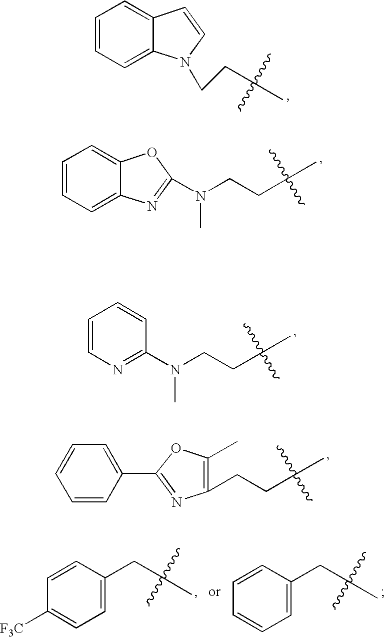
and R₂ is hydrogen or substituted or unsubstituted C₁₋₆ alkyl.

11. An alanine compound or its salt of claim 10 selected from the group consisting of:
(2S)-2-[N-(trans-4-isopropylcyclohexylcarbonyl)amino]-3-[4-[2-(5-methyl-2-phenyl-4-oxazolyl) ethoxy]phenyl]propionic acid;
(2R)-2-[N-(trans-4-isopropylcyclohexylcarbonyl)amino]-3-[4-[2-(5-methyl-2-phenyl-4-oxazolyl)ethoxy]phenyl]propionic acid;
(2S)-2-[N-(trans-4-isopropylcyclohexylcarbonyl)amino]-3-[4-[2-[N-methyl-N-(2-benzoxazolyl)amino]ethoxy]phenyl]propionic acid;
(2R)-2-[N-(trans-4-isopropylcyclohexylcarbonyl)amino]-3-[4-[2-[N-methyl-N-(2-benzoxazolyl)amino]ethoxy]phenyl]propionic acid;
(2S)-2-[N-(trans-4-isopropylcyclohexylcarbonyl)amino]-3-[4-[2-(1-indolyl)-ethoxy]phenyl]propionic acid;
(2R)-2-[N-(trans-4-isopropylcyclohexylcarbonyl)amino]-3-[4-[2-(1-indolyl)-ethoxy]phenyl]propionic acid;
(2S)-2-[N-(trans-4-isopropylcyclohexylcarbonyl)amino]-3-[4-(4-trifluoromethyl-benzyloxy)phenyl]propionic acid;
(2R)-2-[N-(trans-4-isopropylcyclohexylcarbonyl)amino]-3-[4-(4-trifluoromethylbenzyloxy)phenyl]propionic acid;
(2S)-2-[N-(trans-4-isopropylcyclohexylcarbonyl)amino]-3-(4-benzyloxyphenyl)propionic acid;
(2R)-2-[N-(trans-4-isopropylcyclohexylcarbonyl)amino]-3-(4-benzyloxyphenyl)propionic acid;
(2S)-2-[N-(trans-4-isopropylcyclohexylcarbonyl)amino]-3-(4-butoxyphenyl)-propionic acid;
(2R)-2-[N-(trans-4-isopropylcyclohexylcarbonyl)amino]-3-(4-butoxyphenyl)-propionic acid;
(2S)-2-[N-(trans-4-isopropylcyclohexylcarbonyl)amino]-3-(4-ethoxyphenyl)-propionic acid;
(2R)-2-[N-(trans-4-isopropylcyclohexylcarbonyl)amino]-3-(4-ethoxyphenyl)-propionic acid;
(2S)-2-[N-(trans-4-isopropylcyclohexylcarbonyl)amino]-3-(4-methoxyphenyl)propionic acid;
(2R)-2-[N-(trans-4-isopropylcyclohexylcarbonyl)amino]-3-(4-methoxyphenyl)propionic acid;
(2S)-2-[N-(trans-4-isopropylcyclohexylcarbonyl)amino]-3-(4-hydroxyphenyl)propionic acid;
(2R)-2-[N-(trans-4-isopropylcyclohexylcarbonyl)amino]-3-(4-hydroxyphenyl)propionic acid;
(2S)-2-[N-(trans-4-isopropylcyclohexylcarbonyl)amino]-3-[4-[2-[N-methyl-N-(2-pyridyl)amino]ethoxyl]phenyl]propionic acid;
(2R)-2-[N-(trans-4-isopropylcyclohexylcarbonyl)amino]-3-[4-[2-[N-methyl-N-(2-pyridyl)amino]ethoxyl]phenyl]propionic acid;
(2S)-2-[N-(trans-4-isopropylcyclohexylcarbonyl)amino]-3-(4-hydroxyphenyl)propionic acid methyl ester; and
(2R)-2-[N-(trans-4-isopropylcyclohexylcarbonyl)amino]-3-(4-hydroxyphenyl)propionic acid methyl ester.

12. A method for preparing an alanine compound or its salt of claim 10, said method comprising the following steps:
(1) condensing trans-4-isopropylcyclohexylcarboxylic acid N-hydroxylsuccinimide ester and L-tyrosine methyl ester or D-tyrosine methyl ester conduct in an inert solvent to produce 2-[N-(trans-4-isopropylcyclohexylcarbonyl)amino]-3-(4-hydroxyphenyl) propionic acid methyl ester; and
(2) conducting a Mitsunobu reaction with the 2-[N-(trans-4-isopropylcyclohexylcarbonyl)amino]-3-(4-hydroxyphenyl) propionic acid methyl ester and a corresponding heterocycloalkyl alcohol or aromatic alcohol, followed by hydrolyzing the reaction product with inorganic base to obtain the compounds of formula (I), wherein R₁ is 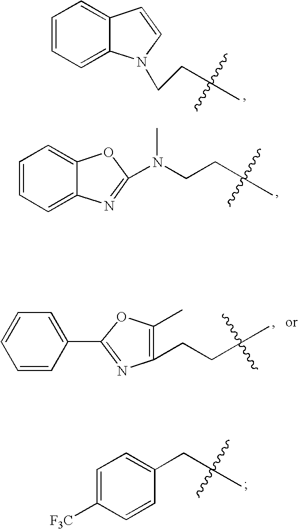
and R₂ is hydrogen; or
(2) esterifying said_{—}2-[N-(trans-4-isopropylcyclohexylcarbonyl)amino]-3-(4-hydroxyphenyl)propionic acid methyl ester with a corresponding alkyl halide under basic condition to obtain the compounds of formula (I), wherein R₁ is 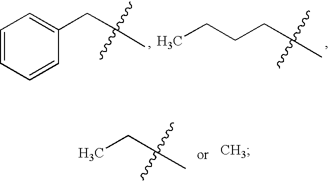
and R₂ is hydrogen ; or
(2) hydrolyzing said_{—}2-[N-(trans-4-isopropylcyclohexylcarbonyl)amino]-3-(4-hydroxyphenyl)propionic acid methyl ester to obtain the compound of formula(l), wherein R₁ and R₂ both are hydrogen; and, optionally
(3) preparing a corresponding pharmaceutical acceptable salt.

13. The method of claim 12, wherein the inert solvent is selected from chloroform, dichloromethane, ether, and tetrahydrofuran.

14. The method of claim 12, wherein the inorganic base of said hydrolyzing step is selected from sodium hydroxide, lithium hydroxide, potassium hydroxide, sodium carbonate, potassium carbonate and lithium carbonate; said hydrolyzing being optionally conducted in the presence of a solvent selected from a mixed solvent of tetrahydrofuran and methanol, a mixture of alcohols solvent, or chloroform, dichloromethane, or benzene.

15. The method of claim 12, wherein said basic condition includes the addition of an inorganic base selected from sodium hydroxide, lithium hydroxide, potassium hydroxide, sodium carbonate, potassium carbonate and lithium carbonate.

16. The method of claim 12, wherein said esterifying is conducted at a temperature between —10-180° C. and optionally in the presence of a solvent selected from N,N-dimethylformamide, DMSO and H₂O and optionally for 1-72 h.

17. A method of preparing a compound of claim 10, comprising the following steps:
(1) condensing_{—}2-[N-(trans-4-isopropylcyclohexylcarbonyl)]-3-(4-hydroxyphenyl)propionic acid methyl ester with an amino-protected 2-methylaminoethanol to form a protected product, deprotecting said protected product, and refluxing with excessive 2-fluoropyridine, and hydrolyzing with a base to obtain a compound of formula (I), wherein R₁ is 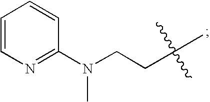
and R₂ is hydrogen; and optionally
(2) preparing a pharmaceutical acceptable of said compound.

18. The method of claim 17, wherein said base is an inorganic base selected from sodium hydroxide, lithium hydroxide, potassium hydroxide, sodium carbonate, potassium carbonate, lithium carbonate; said hydrolyzing being optionally conducted in the presence of a solvent selected from a mixed solvent of tetrahydrofuran and methanol, a mixture of alcohols solvent, or chloroform, dichloromethane, or benzene.

19. A method of treating a person with type II diabetes comprising administering a compound of claim 10 to said person.
